(19) United States
(12) Patent Application Publication
(10) Pub. No.: US 2005/0186565 A1
(43) Pub. Date: Aug. 25, 2005

(54) Method and spectral/imaging device for optochemical sensing with plasmon-modified polarization

(75) Inventor: Henryk Malak, Ellicott City, MD (US)

Correspondence Address: Henryk Malak, 8444 High Ridge Road, Ellicott City, MD, 21043, US

(73) Assignee: American Environmental Systems, Inc.

(21) Appl. No.: 11/117,001

(22) Filed: Apr. 29, 2005

Related U.S. Application Data
(63) Continuation-in-part of application No. 10/656,629, filed on Sep. 8, 2003.
(60) Provisional application No. 60/446,096, filed on Feb. 10, 2003.

(51) Int. Cl. C12Q 1/70; C12Q 1/68; G01N 33/53; C12M 1/34

(52) U.S. Cl. 435/5; 435/6; 435/7.1; 435/287.1

(57) ABSTRACT

The invention discloses a method and spectral-imaging device for optochemical sensing with plasmon-modified multiband fluorescence polarization and with plasmon-modified polarization phase shift changes of a light beam reflected and/or passed through a total internal reflection conducting structure. The optochemical sensing is performed for molecules placed nearby the conducting structure and being excited by surface plasmon resonance (SPR) to lower excited state (LES) and/or to higher excited states (HES). The invention also describes the spectral imaging device with an improved sensitivity of several orders of magnitude. The disclosed method and imaging device may find applications in clinical diagnostics, pharmaceutical screening, biomedical research, biochemical-warfare detection and other diagnostic techniques. The device can be used in bio-chip and micro-array technologies, flowcytometer, fiber optic and other types of diagnostic devices.

### CROSS REFERENCE TO RELATED APPLICATIONS

This application is a continuation-in-part of U.S. patent application Ser. No. 10/656,629, filed Sep. 8, 2003 entitled “Optochemical Sensing with Multi-Band Fluorescence Enhanced by Surface Plasmon Resonance” and U.S. provisional patent application Ser. No. 60/446,096 filed Feb. 10, 2003 entitled “Optochemical Sensing with Multi-Band Fluorescence Enhanced by Surface Plasmon Resonance” each of which is incorporated by reference herein in their entirety.

### STATEMENT REGARDING FEDERALLY SPONSORED RESEARCH

There is NO claim for federal support in research or development of this product.

### FIELD OF THE INVENTION

This invention relates to methods and spectral imaging techniques in conjunction with plasmon-enhanced optical effects and to the use of such methods and techniques for biomedical diagnostics.

### BACKGROUND OF THE INVENTION

Most currently deployed optical biosensing methods are based on detection of fluorescence markers incorporated into a range of biomolecules. These methods require relatively complex sample preparation that can alter the desired results and they suffer from long (20 minutes-2 hours) analysis times with loss of viability. In attempts to avoid these limitations, a separate class of substantially less sensitive biosensors that measures the intrinsic fluorescence of biomolecules or pathogens has been identified based on the fact that the majority of biological specimens include fluorophores as aromatic amino acids, NADH, flavins and chlorophylls/porphyrins. Use of these intrinsic fluorescence-based sensors is currently limited to distinguishing between biological and inorganic samples, and between proteins, NADH, flavins and chlorophylls/porphyrins.

The lack of sensitivity within this long-emergent class of intrinsic biomolecular detection technology has prevented direct intracellular detection and identification of different proteins. The low-excitation state (LES) emission rate for a fluorophore is defined by its natural fluorescence lifetime (as a rule, it does not exceed 10⁹s⁻¹). This value puts a limit on the rate of nonradiative decay and, consequently, the quantum yield (QY) of fluorescence. The nonradiative decay of the high-excited state (HES) is thousands of times faster than radiative decay of this state. The lifetime of the HES emission occurs on a 10⁻¹³−10⁻¹²s time scale. This leads to a very low QY for the HES emission, and difficulties in detection of HES fluorescence. The ratio of QY for LES to HES fluorescence may be as high as 10⁵. It is the low value of QY that prevents the multi-band HES fluorescence from being exploited for its very selective (much more so than LES fluorescence) optical signature.

In the last several years, there has been extensive research related to surface plasmon resonance (SPR)-enhanced fluorescence. Several papers cited here are representative of the scientific results indicating enormous SPR enhancement of fluorescence and decreasing fluorescence lifetimes of low fluorescence quantum yield fluorophores (Lakowicz et al, “Intrinsic fluorescence from DNA can be enhanced by metallic particles”, Biochem. Biophys. Res. Comm. 286, 875 (2001); Gryczynski et al., “Multiphoton excitation of fluorescence near metallic particles: enhanced and localized excitation”, J Phys. Chem. B, 106, 2191 (2002); M. Moskovits: Rev. Mod. Phys. 57, 783 (1985)). However, there are not reports about SPR-enhanced fluorescence polarization of molecules and the feasibility of using SPR-enhanced polarization in diagnostic techniques.

There are also reports related to a plasmon-coupled fluorescence emission technique in which researchers claimed increased fluorescence detection because of more directional fluorescence (Lakowicz et al,“Radiative decay engineering 3. Surface plasmon-coupled directional emission”, Analytical Biochemistry 324, 153-169(2004)). Researchers observed random fluorescence polarization in the p-plane, perpendicular to the direction of fluorescence, and they did not indicate any value of fluorescence polarization for use in polarization diagnostics.

There is also the ellipsometric imaging technique used for sensing molecules placed in an evanescent field under total internal reflection conditions. This technique does not require the use of fluorescent molecules; detection is at low light intensities and the technique can be applied in bio-chip, micro-arrays, and micro-titer technologies. The major disadvantage of this technique is relatively poor sensitivity. There is a great need in diagnostics to enhance sensitivity of the ellipsometric imaging technique by two or three orders of magnitude.

This invention discloses novel approaches in the ellipsometric imaging technique, particularly describes a method of using linearly polarized light instead of elliptically polarized light and/or movement of an analyzer to significantly improve the sensitivity of the device used for diagnostics. An additional increased sensitivity of the device is proposed in this invention by adding fluorescence polarization imaging data and by global analysis of the data obtained from both techniques.

### SUMMARY OF THE INVENTION

The invention discloses a method and spectral-imaging device for optochemical sensing with plasmon-enhanced multiband fluorescence polarization and with plasmon-induced polarization phase shift changes of a light beam reflected and/or passed through a total internal reflection conducting structure. A fluorophore with low fluorescence quantum yield placed nearby the conducting structure can display a few orders of magnitude of enhancement of the absorption and emission rates in the presence of surface plasmon resonance (SPR). The observed enhancement of the rates for lowest excitation state (LES) and higher excitation states (HES) of the molecule is associated with decreasing fluorescence lifetimes of fluorophores and with increasing multiband fluorescence polarization values. The invention also describes novel approaches applied in the ellipsometric imaging technique, and in particular, describes a method of using linearly polarized light instead of elliptically polarized light and/or polarization movements of polarizer and/or analyzer which improve sensitivity of this technique by a few orders of magnitude. The invention also considers the implementation of spectral imaging capabilities to the device. The spectral-imaging device would be capable of registering from each pixel of the sensing area multiband fluorescence polarization values and a phase shift of the excitation light. This multiparametric information will be next globally analyzed by custom designed software. The disclosed method and spectral-imaging device can be applied in clinical diagnostics, pharmaceutical screening, biomedical research, biochemical-warfare detection and other diagnostic techniques. The device can be used in a bio-chip and micro-array technologies, flowcytometer and other types of diagnostic devices.

### BRIEF DESCRIPTION OF THE DRAWINGS

FIG. 1. Schematic diagram of the fluorophore electronic states, processes (left) and fluorescence spectra (right). 1 and 2—one photon absorption/LES and HES population, 1+3—two-photon step wise absorption/HES population, 4—LES fluorescence, 5—HES fluorescence, 6—LES nonradiative decay, 7—HES nonradiative decay.

FIG. 2. Dependence of enhanced fluorescence intensity with nearby silver nanoparticle on fluorophore quantum yield.

FIG. 3. Schematic diagram of the surface plasmon resonance-enhanced multiband fluorescence spectral/imaging optical setup.

FIG. 4. Schematic diagram of the hyperspectral imager (HSI) shown in FIG. 3.

FIG. 5. Absorption and fluorescence spectra of Rhodamine 6 G solution: (1) LES fluorescence, (2) long-wavelength absorption spectrum, (3) short-wavelength absorption spectrum, (4) HES fluorescence. (5) absorption of excited molecules. Upper right: scheme of molecular states and optical processes.

FIG. 6. An imaging polarization device based on a scanned linearly polarized light excitation and a wide-field observation.

FIG. 7. An imaging polarization device based on a scanned elliptically polarized light excitation and a wide-field observation.

FIG. 8. An imaging polarization device based on an expanded beam linearly polarized light excitation and a wide-field observation.

FIG. 9. An imaging polarization device based on an expanded beam elliptically polarized light excitation and a wide-field observation.

FIG. 10. An imaging polarization device based on an expanded beam linearly polarized light excitation and a wide-field observation comprising a spectral active optics.

FIG. 11. An imaging polarization device based on an expanded beam linear or elliptical polarized light excitation and simultaneous wide-field observations of multiband fluorescence polarization and phase shift of excitation light being totally reflected from the metallic structure with nearby molecules and/or analytes.

FIG. 12. A polarization sensing device based on a single mode optical fiber and total internal reflection from a metallic structure for simultaneous observations of multiband fluorescence polarization and phase shift of excitation light being totally reflected from the metallic structure adjacent to nearby molecules and/or analytes.

### DETAILED DESCRIPTION OF PREFERRED EMBODIMENTS

1. Abbreviations and Definitions
SPR—surface plasmon resonance generated in a nanoparticle under illumination by electromagnetic radiation and other forms of energy
one-photon mode of excitation—process in which molecule is excited by a one-photon absorption event
two-photon mode of excitation—process in which a molecule is excited by simultaneous absorption of two photons
multi-photon mode of excitation—process in which a molecule is excited by simultaneous absorption of three or more photons
step-wise mode of excitation—process in which a molecule is excited by absorption of one photon and subsequently by absorption of a second photon
up-conversion mode of excitation—process in which a molecule is excited by a photon whose energy is lower than that of the lowest excited state of the molecule
nanoisland—a nanoparticle on a substrate without defined shape
aerogel—a nanoporous material
quantum dot—a nanoparticle, whose size is a few nanometers and exhibits luminescence properties
LED—light emitting diode
UV light—ultraviolet light
UV—VIS—NIR light—ultraviolet, visible and near-infrared light
multiband absorption—absorptions to a lowest excited state (LES) and to higher excited states (HES) of a molecule
multiband fluorescence—fluorescence from a lowest excited state (LES) and from higher excited states (HES) of a molecule
single band absorption—absorption to any excited state of a molecule
single band fluorescence—fluorescence from any excited state of a molecule
LES—a lowest excited state of a molecule
HES—higher excited states of a molecule
optically nonlinear medium—medium in which absorption of light by medium is
nonlinearly dependent on intensity of light
transient absorption—absorption from the lower excited state to higher excited states of a molecule
recognitive material substance—a material where the external surface is covered with recognitive chemical ligands, immunolabels, phage display or other type recognitive substances
Polarization phase shift—change of polarization state of a light beam
CW optical source—continuous waves source
Fluorescence polarization—an inherent fluorescent property of a fluorophore and is defined by the ratio of fluorescence intensities difference of vertical and horizontal polarizations of fluorescence light in regards to vertical polarization of an excitation light of the fluorophore, to the sum of the total fluorescence intensity emitted by the fluorophore

2. Exemplary Embodiments

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

Current fluorescence techniques, despite their relatively high sensitivity, are restricted by fundamental photo-physical processes. For certain fluorophores, fluorescence might not be sufficiently sensitive to be used for successful identification of single-particle samples. For example, the typical fluorescence spectra of bacteria do not always provide a sufficiently selective signature of pathogens (R. G. Pinnick, et al., “Real-time measurement of fluorescence spectra from single airborne biological particles”, Field Anat. Chem. Technol. 3,221 (1999); Scully et al., “FAST CARS: Engineering a laser spectroscopic technique for a rapid identification of bacterial spores”. PNAS, 99, 10994, (2002)).

The invention provides a novel methodology that overcomes limitations of the conventional fluorescence sensing. To increase the fluorescence intensity, the effect of enhanced fluorophore absorption/emission rates by surface plasmon resonance (SPR) of nearby metal (silver, gold) nanoparticles is employed (M. Kerker, “Optics of colloid silver”, J. Colloid Interface Sci. 105, 298 (1985); Lakowicz et al, “Intrinsic fluorescence from DNA can be enhanced by metallic particles”, Biochem. Biophys. Res. Comm. 286, 875 (2001); Gryczynski et al., “Multiphoton excitation of fluorescence near metallic particles: enhanced and localized excitation”, J. Phys. Chem. B, 106, 2191 (2002)). When the fluorophore is in direct contact with a metal nanoparticle, fluorescence is completely quenched by energy transfer to metal. However, at the distance of 10 nm-100s nm between the fluorophore and metal nanoparticle the absorption and emission rates of the lower excited state (LES) can be, respectively, enhanced by factors of ˜10² and ˜10³ (Lakowicz et al, “Intrinsic fluorescence from DNA can be enhanced by metallic particles”, Biochem. Biophys. Res. Comm. 286, 875 (2001)). The enhancement of the emission intensity depends on fluorescence quantum yield Q, where 0≦Q≦1.

It is the first invention that implements a measurement of multi-band fluorescence for analyte identification in fluorescence polarization sensing and imaging. Current fluorescence sensors are based on a fundamental principle of molecular fluorescence known as the Kasha rule (M. Kasha, “Characterization of electronic transitions in complex molecules”, Discuss Faraday Soc., 8, 14 (1950)). According to the Kasha rule, a fluorophore in the condensed phase emits a single-band spectrum from its lowest singlet excited state (LES), due to the vibrational relaxation and non-radiative dissipation of excitation energy. The natural emission rate for a fluorophore (<10⁹ s⁻¹) is defined by fluorophore transient dipole puts a limit on a rate for fluorophore nonradiative decay of measured fluorescence.

Fluorescence from high-excited state (HES) can expand LES fluorescence information about the molecular structure of an analyte in question. However, the non-radiative decay of the high-excited state is thousands of times faster than HES radiative decay, which leads to a very low Q for the HES emission (much lower than for the LES emission) and difficulties in detection of HES fluorescence. The ratio of Q for LES to HES fluorescence may be as high as 10⁵ (Bogdanov, “Fluorescence and multiwave mixing induced by photon absorption of excited molecules”, Topics in Fluorescence Spectroscopy, Vol. 5: Nonlinear and Two-photon induced Fluorescence, Ed. J. Lakowicz, Plenum Press, 1997; Galanin et al. “Fluorescence from the second excited electronic level and absorption by excited R6G molecules”, Bull. Acad Sc., Phys. Ser. 36, 850 (1972)).

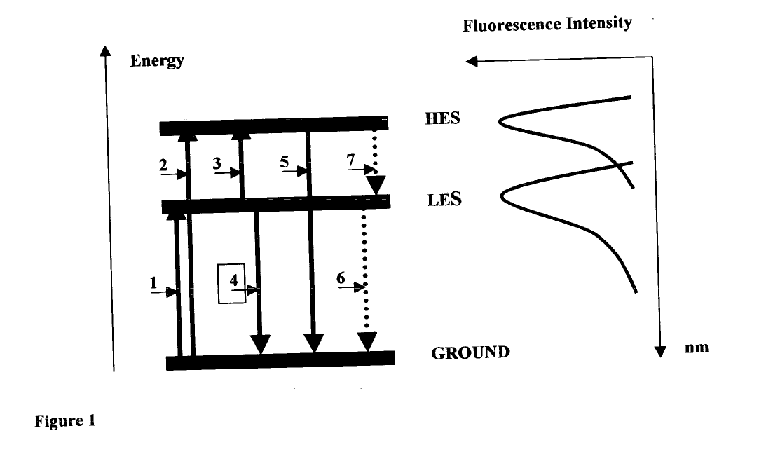

Although HES fluorescence is not available in current fluorescence polarization sensing and imaging, its characteristics have sensitivity to both the excitation energy and the fluorophore's chemical environment. The normally low value of Q prevents the multi-band HES fluorescence from being used as a very selective optical signature. FIG. 1 shows fluorophore electronic states and origin of LES and HES fluorescence spectra.

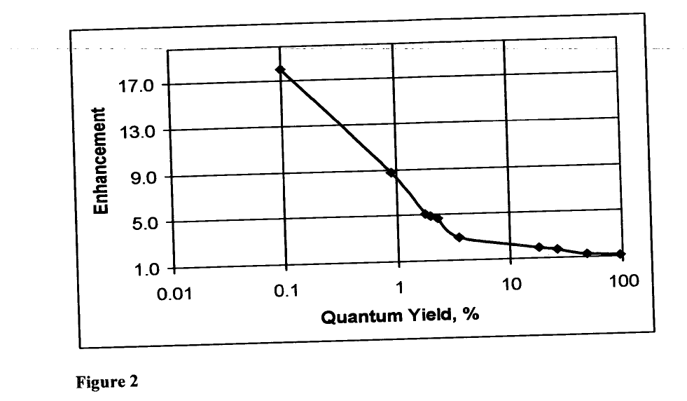

It is proposed by the invention that polarization measurement of multiband, LES and HES fluorescence enhanced by nearby metal nanoparticles can be used as a novel method to detect an optical signature of sensor and/or analyte, and can be used successfully in fluorescence polarization diagnostics. The invention is based on the previously discussed low Q values for non-enhanced HES fluorescence and observed dependence of fluorescence intensity and fluorescence polarization enhance effect on fluorophore quantum yield. In a recent experiment, the emission intensity measured for a series of fluorophores in the vicinity of metal nanoparticles was greatly increasing for the decreased values of Q for fluorescence (FIG. 2). This result is consistent with findings observed by Lakowicz et al., noting a substantial intrinsic fluorescence enhancement for DNA (Q≈0.01%) at room temperature (J. R. Lakowicz et al, “Intrinsic fluorescence from DNA can be enhanced by metallic particles”, Biochem. Biophys. Res. Comm. 286, 875 (2001)). Without SPR-mediated enhancement, the DNA fluorescence could not be observed at room temperature. Thus, SPR-mediated fluorescence enhancement is a quantum yield dependent effect. The SPR-enhanced fluorescence is associated with decreasing fluorescence lifetimes of fluorophores. Shorter fluorescence lifetimes of fluorophores provide higher fluorescence polarization of these fluorophores.

Because quantum yield and lifetime for HES and LES fluorescence of the same fluorophore differ by orders of magnitude, the enhancement effect is expected to be high for a short-living HES (low Q) and low for a long-living LES (high Q) of the same fluorophore. As a result, fluorescence intensities from HES and LES would reach comparable levels. The short-living HES has a very high polarization value. Therefore, HES fluorescence polarization could then be used as a new measurable optical signature and also be used as a polarization sensor in diagnostics. The long-living LES is also SPR-enhanced which leads to shortened fluorescence lifetimes and increasing fluorescence polarization of LES emissions. Hence, SPR-enhanced emissions from both the LES and HES provide increased fluorescence polarization/anisotropy of fluorophore which can significantly increase the sensitivity of polarization sensing and polarization imaging. The invention considers the use of fluorescence intensity and fluorescence polarization from LES of fluorophore as the part of this invention.

In addition to better specificity, the polarization sensor proposed in this invention is superior to conventional polarization sensors in polarization dynamic sensitivity. It is the result of an enhanced fluorophore absorption rate with nearby metal nanoparticles by SPR interactions with fluorophore. Absorption rate enhancement is caused by the electro-magnetic (EM) field E generated by surface plasmons in the evanescent zone. The magnitude of the SPR-enhanced EM field exceeds the magnitude of the EM field of incident light by 10² fold. Since the absorption rate of the one-photon excitation is proportional to |E|², the absorption rate can be enhanced by ˜10⁴ compared to the absorption rates of sensors that do not employ SPR. The SPR EM fields force fluorophore to anisotropic emission, like stimulated emission in which fluorescence is highly polarized. It is also possible that SPR EM fields may induce new electromagnetic dipoles in fluorophore that was demonstrated by the inventor in his other research (Optically-Induced Birefringence in Aqueous Hemoglobin Solutions. Z. Blaszczak and H. Malak (1986). Acta Physica Polonica A69:621-629). The SPR-induced rearrangement of electromagnetic dipoles in the fluorophore may lead to enhanced fluorescence, shorter fluorescence lifetime and to a different fluorescence polarization value of the fluorophore. The SPR can enhance or decrease the fluorescence polarization value of fluorophores. In the majority of fluorophores SPR-enhancement is expected. However, some fluorophores with low Q may show either decreased or enhanced fluorescence polarization values for LES and HES fluorescence bands. Enhanced EM fields by surface plasmon are especially effective in non-linear, multi-photon excitation. For a two-photon excitation the absorption rate enhancement could be as high as 10⁸ (J. R. Lakowicz, Y. Shen, S. D'Auria, J. Malicka, J. Fang, Z. Gryczynski, and I. Gryczynski, “Radiative Decay Engineering. 2. Effects of Silver Island Films on Fluorescence Intensity, Lifetimes, and Resonance Energy Transfer”, Anal. Biochem., 301:261 (2002)).

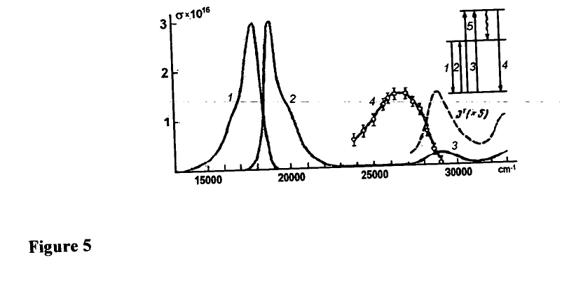

Metal nanoparticles can also enhance the rate of transient absorption by excited fluorophores in a resonant two-photon HES excitation (M. D. Galanin, and Z. A. Chizhikova, “Fluorescence from the second excited electronic level and absorption by excited R6G molecules”, Bull. Acad Sc., Phys. Ser. 36, 850 (1972)). The first photon excites the long-living LES and then, the second photon populates the HES through the SPR-enhanced absorption. Such a step-wise, two-photon HES excitation (M. D. Galanin, and Z. A. Chizhikova, “Fluorescence from the second excited electronic level and absorption by excited R6G molecules”, Bull. Acad Sc., Phys. Ser. 36, 850 (1972)) is then followed by the SPR-enhanced emission. The resonance- and SPR-enhanced two-photon excitation will greatly increase the fluorescence intensity and fluorescence polarization of the fluorophore. This is our concept behind the proposed in the invention multi-signature (HES+LES bands) SPR-enhanced fluorescence intensity and fluorescence polarization. Two-photon, step-wise HES excitation has been shown to generate a measurable intensity of HES emission and to reduce the background contribution (M. D. Galanin and Z. A. Chizhikova, “Fluorescence from the second excited electronic level and absorption by excited R6G molecules”, Bull. Acad. Sc., Phys. Ser. 36, 850 (1972); Lin, and M. R. Topp, “Low quantum-yield molecular fluorescence: excitation energy dependence and fluorescence polarization in xanthene dyes”, Chem. Phys. Lett. 47, 442 (1977)). An example of HES emission spectrum measured at step-wise excitation of Rhodamine 6G (R6G) solution is shown in FIG. 5. The position of the short-wavelength HES fluorescence band correlates with the position of the absorption band but there is no strict mirror symmetry for these bands. This lack of symmetry is caused by the short HES fluorescence lifetime (0.2 ps for R6G), as the HES decay competes with vibrational relaxation of excited fluorophores (M. D. Galanin and Z. A. Chizhikova, “Fluorescence from the second excited electronic level and absorption by excited R6G molecules”, Bull. Acad. Sc., Phys. Ser. 36, 850 (1972).

This invention also applied to a multiparametric polarization sensing and polarization imaging of analytes by using plasmon-enhanced multiband fluorescence with other techniques like surface enhanced multiband Raman scattering, surface plasmon coupled emission, total internal reflection, fluorescence resonance energy transfer, fluorescence resonance energy transfer depolarization, metal-enhanced fluorescence from HES and/or LES, SERS, one-photon excitation, multi-photon excitation, step-wise excitation, phase contrast microscopy, polarization phase shift of excitation light, holographic microscopy, multiphoton microscopy, interference contrast, but not limited to them.

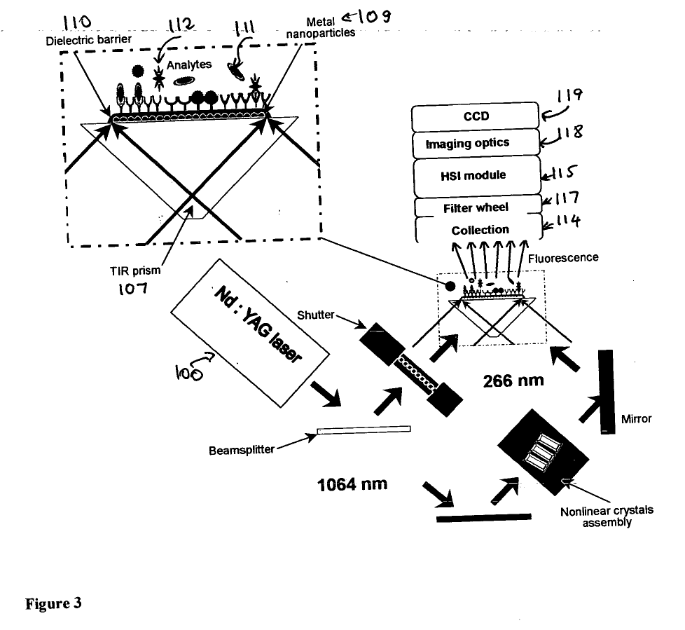

Hyperspectral imaging is a preferable method to measure and analyze the fluorescence of analytes immobilized on a sensor surface. FIG. 3 shows a possible schematic of proposed sensor. Metal nanoparticles (tens of nanometers in diameter) 109 are placed on the surface of glass substrate (prism) 107. Nanoparticle layer 109 is coated with a 10-100s nm thick dielectric layer (polymer or SiO₂) 110 to create a physical barrier between the metal particles and a fluorophore. Microarray of analyte 112 captured spots is attached to a surface of dielectric layer 110. The excitation can be delivered via evanescent wave coupling using the effect of total internal reflection at the prism surface 107.

OPTICAL SET-UP. The entire microarray can be illuminated with laser pulses 100 at two different wavelengths. To produce both LES and HES signatures, the sample is simultaneously illuminated by two nanosecond laser pulses at different wavelengths, for example, 4^{th} harmonics (266 nm) and fundamental (1064 nm) wavelength of Nd:YAG laser. The conventional (LES) fluorescence spectrum will be acquired following single-photon excitation at 266 nm To obtain HES fluorescence spectrum, two-photon resonant (step-wise) excitation is used. In the first step, LES is populated when the molecules in their ground electronic state absorb a photon at 266 nm. In the second step the excited molecules in LES absorb the second photon at 1064 nm; this results in population of HES. The measured HES fluorescence spectrum is blue-shifted compared to the LES fluorescence. To obtain the full analyte optical signature (LES+HES fluorescence), many other combinations can be used in the step-wise excitation. A Nd:YAG laser 100 equipped with a standard set of nonlinear crystals can generate pulses at the fundamental frequency plus four harmonics.

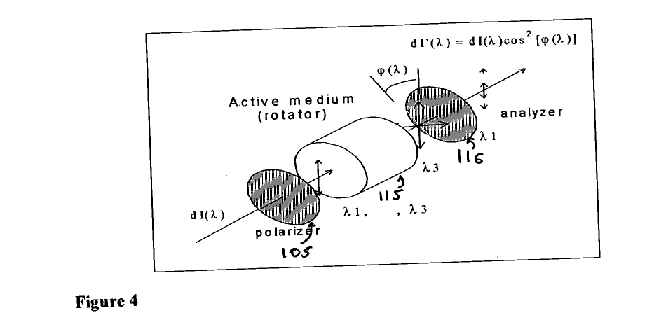

In this example, the output, a Q-switched Nd:YAG laser (5 ns pulses, up to 100 Hz repetition rate), consists of the fundamental (1064 nm) and 2^{nd} harmonics (532 nm) and/or 3^{rd} harmonics (355 nm), and/or 4^{th} harmonics (266 nm). This multitude of wavelengths provides a high degree of flexibility in the detection of practically any organic/inorganic matter. The fundamental output is divided into two beams by means of a 60/40 beam splitter. The 40% fraction of the 1064 nm beam passes through an assembly of nonlinear crystals and is converted into the harmonics which are directed into the total internal reflection (TIR) prism 107 made of fused silica The harmonics illuminate the glass-sensor interface at the critical angle and excite the bio-agent fluorophores attached (captured) to the microarray via evanescent wave illumination. The remaining 60% of the fundamental (1064 nm) enters the TIR prism 107 from the opposite prism side and overlaps with the harmonics beam at the glass-sensor interface. A shutter placed in the fundamental beam controls the excitation scheme by blocking passing the 1064 nm radiation. Microarray emission is collected by infinity-corrected lens 122 and transmitted through laser cut-off filter 117 and the spectral active optics 115. An imaging lens 118 produces a microarray spectral image which is then captured by a cooled CCD array 119. FIG. 4 shows a hyperspectral imager (HSI) employing the effect of slit-free, optical-rotation dispersion on polychromatic radiation (P. Herman et al. “Compact hyperspectral imager for low light applications” SPIE Proc. 2001, 4259, pp. 8-16). This module utilizes the effect of optical rotation dispersion (ORD) on polychromatic light. It consists of a polarization rotator—an optically active medium (crystalline quartz) 115 placed between a pair of polarizers (105 and 116) with their transmission axes aligned parallel to each other. The polarization direction of linearly polarized input light rotates during propagation through the rotator and the rotation angle depends on the wavelength and the rotation power of the optical rotator. Due to the ORD effect, the polarization planes of different spectral components become angularly dispersed after passage through the rotator. The emerging light is partially blocked by the output polarizer and the attenuation of the light at different wavelengths is determined by the material-dependent ORD function. Each wavelength component contributes to every point in the image according to the cosine square of the angle between the polarization of the rotated wavelength component and the fixed output polarization analyzer. The optically active medium 115 can also be liquid crystal, optical modulator or other spectrally active optics.

However, other optical techniques can be also applied with optochemical multiband enhanced fluorescence polarization sensing, like time-resolved spectroscopy, fluorescence polarization, fluorescence recovering after photobleaching, fluorescence resonance energy transfer, fluorescence resonance energy transfer depolarization, enhanced multiband Raman scattering (but not limited to them). Thus, hyperspectral detection and other above mentioned techniques could be used for optochemical sensing employed multi-band enhanced emission.

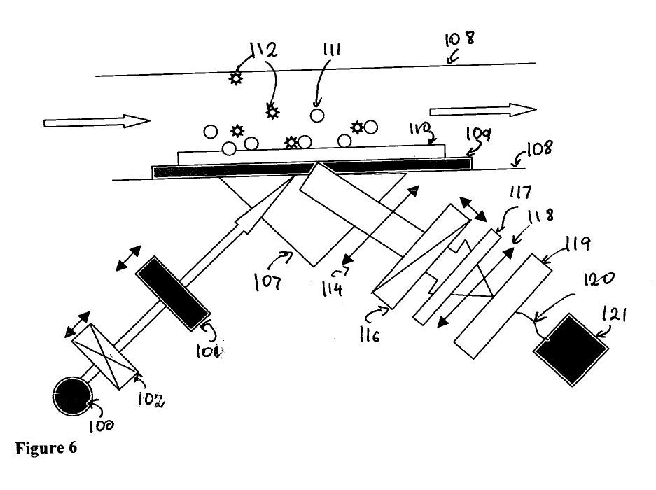

The multi-band enhanced emission can be generated by an electromagnetic radiation source in single, and multi-photon and/or nonlinear optical modes of excitation. It can also be generated by chemiluminescence, electro-optically, electrochemically and other luminescence and non-luminescent techniques. In all of these methods, band-selective intensity-enhancement leads to comparable intensity HES and LES bands. One of the embodiments of the invention is a spectral imaging device for measuring SPR-enhanced multiband fluorescence polarization and polarization phase shift of the excitation light reflected or passed through the conducting structure with nearby molecules and analytes. FIGS. 6-12 show different designs of this device. The device shown in FIG. 6 is a laser scanning device with linearly polarized excitation and a wide-field spectral and polarized observation. The major components of the device are: a SPR laser source 100, polarization rotator 101, x-y light scanning component 102, prism 107, substrate 108 covered with conducting structure 109 and biochemical or dielectric spacer 110, analyzer 116, composition of spectral filters 117, two-dimensional detector 119 with computer connection 120 and computer 121. The molecules 111 and/or analytes 112 are placed in an SPR-enhanced evanescent excitation field. Some applications may require micro or sub-micro imaging resolutions, therefore the device can be further provided with a microscope objective 114 and an imaging lens 118 placed in an observation path. The composition of spectral filters 117 can select observation of multiband fluorescence or excitation light. The filters can be selected mechanically or electro-optically. The preferable choice of the spectral filters is liquid crystal tunable optical filter, in which the wavelength of the filter can be changed in a millisecond or less. The device can perform spectral imaging by changing the position of the analyzer 116 between 0 to 360 degrees with p- or s-polarization of the excitation laser beam to the surface of the conducting structure 109. The images of LES and/or HES polarization fluorescence and phase shifts of excitation light can be analyzed together or separately with custom-designed software.

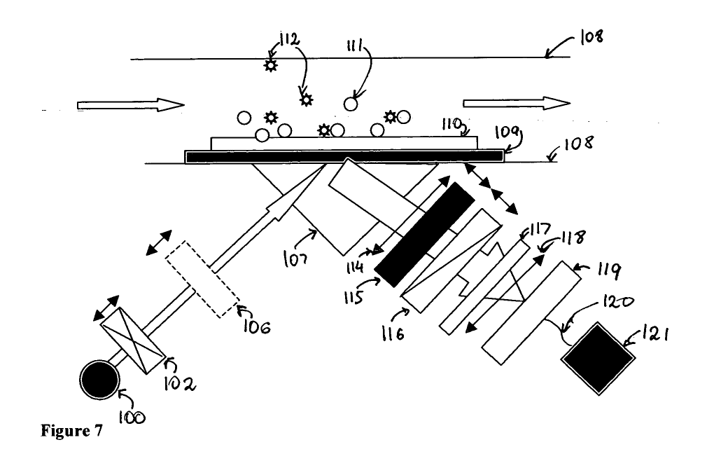

The device can also use elliptically polarized excitation light by adding a phase shifter 106 in an excitation path, as is shown in FIG. 7. However, the elliptically polarized light lowers the detection dynamic range of the polarization values of multiband polarization fluorescence and phase shifts. Hence, the device with phase shifter 106 would be less sensitive, but is contemplated to be a part of this invention.

The device can also perform hyperspectral imaging by placing in the observation path spectral active optics 115, such as crystalline quartz or liquid crystal or other spectrally sensitive optical component.

Please note that double arrows placed above optical components in FIGS. 6-12 indicates the possibility of movement of these components, such as for example the polarization plane movement of the polarizer 105 and analyzer 116 from 0 to 360 degrees.

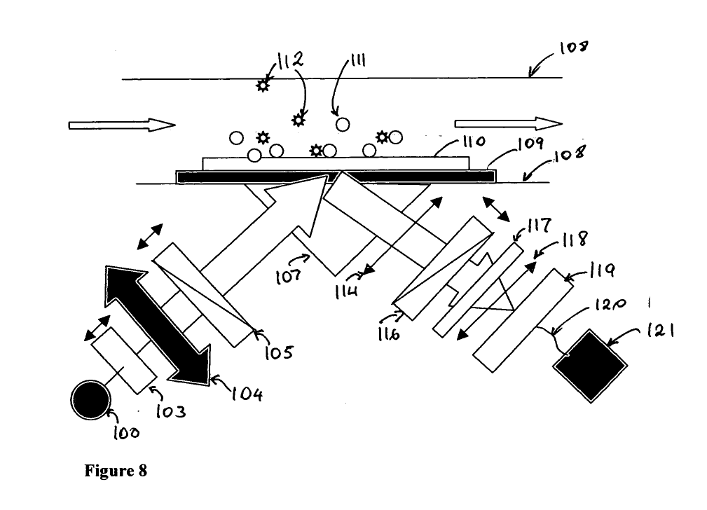

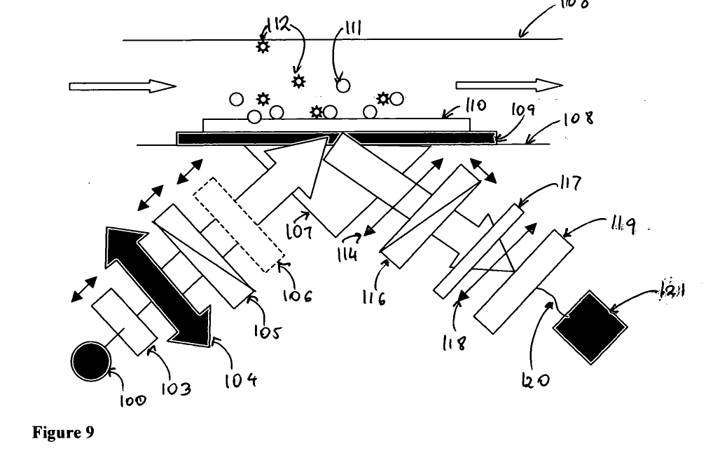

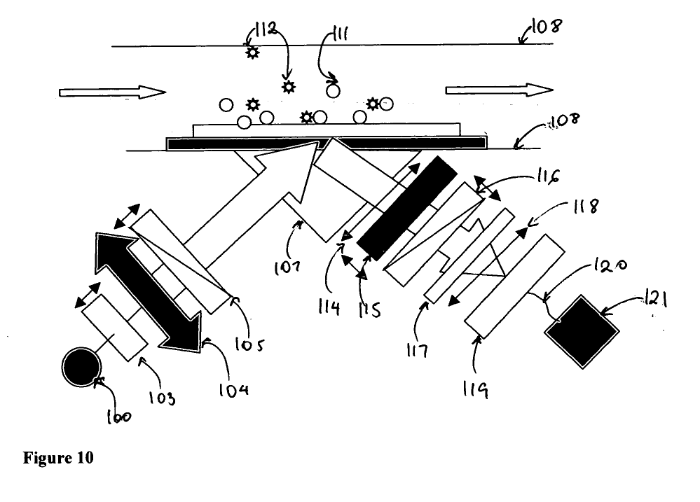

Another embodiment of this invention uses a design of a device shown in FIG. 8. The design is based on an expanded field of excitation and the wide-field of observation. The device has additionally implemented a beam expanding optics 104 in the excitation path. The light source used in this design can be coherent or incoherent, therefore there is also a polarizer 105 in the excitation path. The device can also perform imaging with elliptically polarized light excitation by placing the phase shifter 106 in the excitation path (FIG. 9). FIG. 10 shows additionally the spectral active optics 115 placed in the observation path which allows the device also to perform hyperspectral imaging.

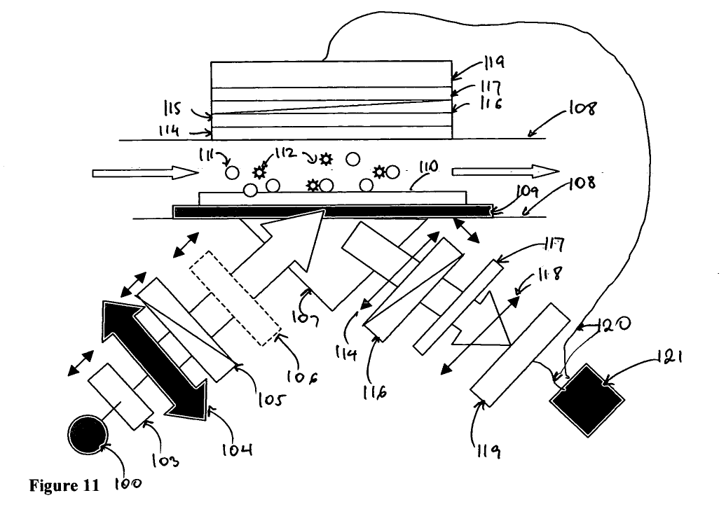

The invention also uses a design shown in FIG. 11 for spectral imaging of multiband fluorescence polarization and phase shift of excitation light after interaction with the conducting structure and nearby molecules and analytes. In that design, measurements of fluorescence polarization and phase shift of the excitation light are performed in different paths and results are sent to the computer and globally analyzed by custom-designed software.

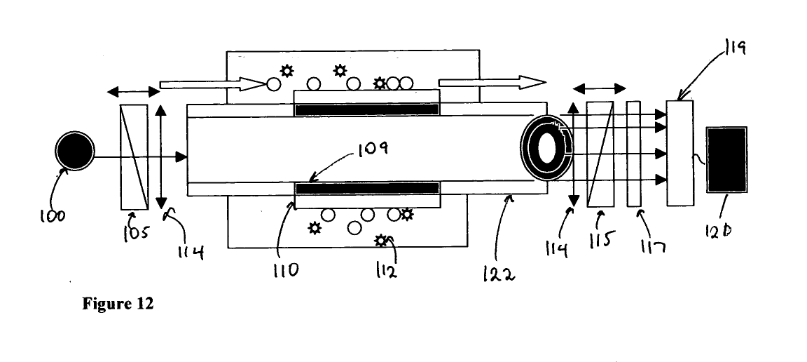

Another embodiment of this invention uses a design of a device shown in FIG. 12. The design is based on a single mode optical fiber 122 in which some part of clad was replaced by a conducting structure 109 and chem-bio recognitive surface or dielectric spacer 110. The linearly polarized light interacting with the conductive structure and adjacent fluorophores and/or analytes will excite fluorophores in the evanescence field and fluorescence emission will reenter back to the fiber 122 and can be imaged at the end of the fiber. The spatial distribution of fluorescence depends on wavelengths carried through the fiber. The single mode fiber should preserve polarization values for relatively narrow spectral distribution. Therefore, spectral and polarization diagnostics can be performed. The phase shift of excitation light can also be measured in the light coming out from the fiber 122.

Regarding a two-dimensional detector 119 used in the designs shown in FIGS. 6-12, it is preferable to use a CCD camera, however different detectors like diodes arrays, micro-channel PMTs, dosimeters or other detectors are also contemplated in this invention.

Another embodiment of the invention discloses a movement of the analyzer 116 in order to increase sensitivity of the device. For each position of analyzer 116 from 0 to 360 degrees, the device measures an intensity image and next analyze of all these images which provides better accuracy and sensitivity of the device. It is expected that using this technique will improve the accuracy of measurements, e.g. sensitivity of the device can be several orders of magnitude higher than in the method based on angular displacement.

Another embodiment of the invention discloses a movement of the polarizer 105 in addition to the movement of the analyzer 116 for the purpose of further sensitivity improvement of the device. The movement of the polarizer changes the polarization of light from p-polarization to s-polarization in relation to the total internal reflection surface. Both polarizations provide different phase shifts, which can be used to further increase the sensitivity of the device. Therefore, this embodiment considers several positions of the polarizer and analyzer with respect to each other and different movement combinations of the polarizer and analyzer with respect to each other.

The invention also considers the use of the phase shifter 106 in the device. The phase shifter can be placed in an excitation path before light is reflected by total internal reflection surface or can be placed in a reflected path. The phase shifter may introduce controlled polarization phase shifts in the excited or reflected light which then can be measured by polarization sensitive detection. The introduced phase shift to imaging or sensing can be additionally supported by controlled movements of the polarizer and/or the analyzer to further improve the sensitivity of the device. The controlled movement of the phase shifter, polarizer and analyzer can be mechanical movements with step motors or can be electro-optical, without moving parts, but is not limited to these options.

Any-one skilled in the art would appreciate spectral capabilities of the device disclosed in the invention. Multiband fluorescence of fluorophores has well spectrally-separated HES and LES fluorescence bands that can be measured using spectral filters 117. The polarization shift of the excitation light technique also uses a spectral band pass filter at wavelength of excitation light. Therefore, both techniques applied together require at least three spectral filters. The use of a prism or a grating in the device is also considered, particularly for sensing. However, in spectral polarization imaging, the prism or the grating may introduce difficulties in getting a high quality image. The invention discloses a spectral polarization sensing with a spectral active optics that can be inserted to the device as is shown in FIG. 4. The spectral active optics can be made from material dispersing colors at different rotation angles and the analyzer and detector can sense such colors and retrieve them in spectral analysis. Spectral polarization imaging can be performed in the following scenarios, but not limited to them. In Scenario I, measurements are first performed by rotation of the analyzer from 0 to 180 degrees when the spectral active optics is set on the position “0”, e.g. is not introduced color rotation dispersion, and next the spectral active optics will introduce color rotation dispersion and the analyzer will perform measurements by rotation from 0 to 180 degrees. The analyses will retrieve colors and polarization in each pixel of the image. In Scenario II, measurements are performed by rotation of the analyzer from 0 to 180 degrees when the spectral active optics is set on the position “0”, and next the analyzer will be set at position “0”, e.g. parallel polarization to polarization of excitation light, and the spectral active optics will rotate colors by 360 degrees and more by changing dispersive values for which the detector records the signal in the form of a rotogram. Subsequent analysis will retrieve polarization and spectral information for each pixel in the image.

The above described method of increased sensitivity of the device applies also to the device which does not has the conductive structure, e.g. any phase shift measurements under total internal reflection conditions using this method are part of this invention.

The conductive structure in the invention can be made of a material selected from the group of silver, silver oxide, silver ion, silver nitrate, ruthenium, platinum, palladium, cobalt, rhenium, rhodium, osmium, iridium, copper, aluminum, aluminum oxide, aluminum alloy, zinc, zinc oxide, nickel, chromium, magnesium, magnesium oxide, tungsten, iron, palladium, gold, titanium, titanium oxide, titanium dioxide, alkaline earth metal, selenium, cadmium, vanadium, vanadium oxide, molybdenum.

The conductive structure can be a thin film, colloid, fiber, metal island, nanowire, nanotube, aerogel, empty shell, shell filled with a conducting material, shell filled with a dielectric material, shell filled with a magnetic material.

The device described in the invention can be built for bio-chip and micro-array technologies, micro-titer plate, cell flow, flow cytometer, micro- or nano-fluidic device, sensor based on single mode fiber, 2-dimensional gel chromatographer, endoscope, total internal reflection device, transmission or reflective microscope, optical digital microscope, holographic microscope, spectrofluorometer.

The method and device can be applied to proteomics, cellomics, genomics, molecular diagnostics, immunological diagnostic, chemical diagnostics, biological diagnostics, spectral and polarization light diagnostics, tissue and cell diagnostics, live tissue and live cell diagnostics, tissue and cell drug diagnostics, cancer diagnostic, bio-warfare agent detection, chemical-warfare agent detection, clinical diagnostic, bacterial and viral detection, biological assay, clinical assay, biomedical research and applications, pharmaceutical research and applications.

It will be understood by those skilled in the art that the present invention is a novel and useful method for highly specific, sensitive and fast optochemical fluorescence polarization sensing and polarization shift imaging.

1. A method and a spectral-imaging device for optochemical sensing with surface plasmon resonance-modified multiband fluorescence polarization and a polarization phase shift of a light beam reflected and/or passed through a total internal reflection conducting structure comprising of:
a. a medium,
b. a conducting structure interfacing with said medium,
c. a molecule placed in said medium and nearby said conducting structure,
d. an analyte placed nearby said molecule and nearby said conducting structure,
e. a spacer to separate said molecule and said analyte from said conducting structure,
f. a surface plasmon resonance source generating surface plasmon resonance in said conducting structure and interacting with said molecule and/or said analyte,
g. a spectral-imaging device for optochemical sensing with surface plasmon resonance-modified multiband fluorescence polarization and a polarization phase shift of a light beam reflected and/or passed through a total internal reflection conducting structure comprising of: said medium, said conducting structure, said molecule, said analyte, said surface plasmon resonance source with a light diffuser, beam expending and collimating optics, a polarizer, an analyzer, an optical prism, collecting optics, a spectral active optics, a set of spectral filters, an one- or two-dimensional spectral sensitive array detector, a controlled electro-optical and/or mechanical hardware, a custom-designed software and computer, a power supply.

2. The method and device of claim 1, wherein said medium is an organic substance, inorganic substance, liquid, gas, solid state material, liquid crystal, biomaterial, live biomaterial, live human or animal body.

3. The method and device of claim 1, wherein said conducting structure is made of a metal selected from the group of silver, silver oxide, silver ion, silver nitrate, ruthenium, platinum, palladium, cobalt, rhenium, rhodium, osmium, iridium, copper, aluminum, aluminum oxide, aluminum alloy, zinc, zinc oxide, nickel, chromium, magnesium, magnesium oxide, tungsten, iron, palladium, gold, titanium, titanium oxide, titanium dioxide, alkaline earth metal, selenium, cadmium, vanadium, vanadium oxide, molybdenum.

4. The method and device of claim 1, wherein said conducting structure has a size in at least one of its dimensions within a range of 1 nm to 100,000 nm.

5. The method and device of claim 3 and claim 4, wherein said conducting structure is a thin film, colloid, fiber, metal island, nanowire, nanotube, aerogel, empty shell, shell filled with a conducting material, shell filled with a dielectric material, shell filled with a magnetic material.

6. The method and device of claim 1, wherein said spacer coating said conducting structure has at least one layer made of a chemorecognitive ligand, biorecognitive ligand, biomolecule, polymer, light sensitive polymer, environmentally sensitive polymer, silicon dioxide, semiconducting material, superconducting material, conducting material, dielectric material.

7. The method and device of claim 1, wherein said molecule is in direct contact with said conducting structure or said molecule is separated from said conducting structure by said spacer of thickness within a range from 0.1 nm to 10,000 nm.

8. The method and device of claim 1, wherein said molecule is a biomolecule, protein, amino acid, peptide, oligonucleotide, lipid, sugar moiety, purine or pyrimidine, nucleoside or nucleotide, genetically engineered biomolecule, bacteria, virus, live cell, abnormal cell, live tissue, fluorescence substance, non-fluorescence substance, phosphorescence substance, marker, biomarker, metal ligand charge transfer complex, up-converted fluorophore, dendrimer, quantum dots, quantum well, carbon nanotube, metal nanoparticle, dielectric nanoparticle, semiconductor nanoparticle, pair of fluorescent donor and fluorescent acceptor, pair of fluorescent donor and quencher, fluorescent nanoparticle.

9. The method and device of claim 1, wherein said surface plasmon resonance source is a linearly polarized electromagnetic source, elliptically polarized electromagnetic source, unpolarized electromagnetic source, nonlinear excitation electromagnetic source, multi-wavelength electromagnetic source, electric source, electrostatic source, magnetic source, sonic source, heat source.

10. The method of claim 9, wherein said electromagnetic source is a CW laser, laser diode, pulsed laser, inorganic or organic light emitting diode, super luminescent diode, lamp, fluorescence source, phosphorescence source, electroluminescence source, chemiluminescence source, bioluminescence source, X-Ray source, ionizing radiation source.

11. The method of claim 10, wherein said electromagnetic source generates a single wavelength or multiple wavelengths within a range of 0.001 nm to 20,000 nm.

12. The method and device of claim 1, wherein said analyte interacts with said molecule and said analyte is selected from the group of a biomolecule, protein, amino acid, oligonucleotide, lipid, purine or pyrimidine, nucleoside or nucleotide, bacteria, virus, sugar moiety, glucose, calcium, sodium, potassium, oxygen, nitrogen, nitric oxide, carbon dioxide, carbon monoxide, hydrogen, chemical substance in solid state, chemical substance in liquid state, chemical substance in gaseous state, inorganic molecule, organic molecule.

13. The method and device of claim 1, wherein said custom-designed software analyzes plasmon-modified multiband fluorescence polarization data and/or plasmon-modified polarization phase shift data for diagnostic purposes.

14. The method and device of claim 1, wherein said optochemical sensing comprises measurements and analysis of said plasmon-modified multiband fluorescence polarization and/or said plasmon-modified polarization phase shift, surface plasmon coupled emission, metal-enhanced fluorescence from said lowest excited state and/or from said higher excited states, SERS from lowest excited state and/or from said higher excited states.

15. The method and device of claim 1, wherein said spectral active optics is a phase shifter with a controlled phase shift between two orthogonal components of light passing through said phase shifter, a color dispersive material with a controlled rotation dispersion of colors, said phase shifter with controlled phase shift between two orthogonal components of light passing through said phase shifter and said colors dispersive material with controlled rotation dispersion of colors, liquid crystal, photonic crystal, birefrengent optics, optical modulator.

16. The method and device of claim 1, wherein said conducting structure is illuminated under total internal reflection conditions by said surface plasmon resonance source with p-polarization of light to a surface of said conducting material, s-polarization of light to a surface of said conducting structure, elliptical polarization of light to a surface of said conducting structure with a controlled phase shift of light by said phase shifter placed in an excitation path.

17. The method and device of claim 1, wherein plasmon-modified multiband fluorescence of said molecule and said light beam reflected and/or passed through the total internal reflection conducting structure is directed to said detector through said collective optics, spectral active optics, analyzer with controlled polarization position and spectral filters.

18. The method and device of claim 1, wherein said optochemical sensing with plasmon-modified multiband fluorescence polarization and/or plasmon-modified polarization phase shift is performed by rotation of said analyzer between 0 to 360 degrees with set position of said spectral active optics and set polarization position of said polarizer, by rotations of said analyzer and said polarizer between 0 to 360 degrees with set position of said spectral active optics, by changing positions of said spectral active optics with set polarization positions of said polarizer and said analyzer, by changing positions of said spectral active optics and by rotation of said analyzer and said polarizer between 0 to 360 degrees.

19. The method and device of claim 1, wherein said device is used for diagnostics in combination with a bio-chip, micro-array, micro-titer plate, cell flow, flow cytometer, micro- or nano-fluidic device, fiber sensor, gel chromatographer, endoscope, total internal reflection device, microscope, spectrofluorometer.

20. The method and device of claim 1 is applied to proteomics, cellomics, genomics, molecular diagnostics, immunological diagnostic, chemical diagnostics, biological diagnostics, spectral and polarization light diagnostics, tissue and cell diagnostics, live tissue and live cell diagnostics, tissue and cell drug diagnostics, cancer diagnostic, bio-warfare agent detection, chemical-warfare agent detection, clinical diagnostic, bacterial and viral detection, biological assay, clinical assay, biomedical research and applications, pharmaceutical research and applications.
